(19) United States
(12) Patent Application Publication
(10) Pub. No.: US 2022/0135506 A1
(43) Pub. Date: May 5, 2022

(54) METHANOL PRODUCTION PROCESS

(71) Applicant: ENI S.p.A., Roma (IT)

(72) Inventors: Sivadinarayana CHINTA, Sugar Land, TX (US); Miasser AL-GHAMDI, Bengaluru (SA); Atul PANT, Bengaluru (IN); Ravichander NARAYANASWAMY, Anekal Taluk Bengaluru (IN); Saud AL-HAGBANI, Thuwal (SA); Arwa RABIE, Tomball, TX (US)

(21) Appl. No.: 17/424,714

(22) PCT Filed: Jan. 21, 2020
(86) PCT No.: PCT/US2020/014395

§ 371 Date: Jul. 21, 2021

Related U.S. Application Data
(60) Provisional application No. 62/794,783, filed on Jan. 21, 2019.

(51) Int. Cl. C07C 29/151; C01B 3/38; C07C 29/80

(52) U.S. Cl. CPC C07C 29/1518; C01B 2203/061; C07C 29/80; C01B 3/386

(57) ABSTRACT

A process for producing methanol including the following steps: (a) reacting, via a catalytic partial oxidation (CPO) reaction, a CPO reactant mixture (hydrocarbons, oxygen, and optionally steam) in a CPO reactor to produce syngas; wherein the CPO reactor has a CPO catalyst; and wherein the syngas includes hydrogen, carbon monoxide, carbon dioxide, water, and unreacted hydrocarbons; (b) introducing the syngas to a methanol reactor to produce a methanol reactor effluent stream; wherein the methanol reactor effluent stream includes methanol, water, hydrogen, carbon monoxide, carbon dioxide, and hydrocarbons; and (c) separating the methanol reactor effluent stream into a crude methanol stream and a vapor stream. The crude methanol stream comprises includes methanol and water. The vapor stream includes hydrogen, carbon monoxide, carbon dioxide, and hydrocarbons; and wherein the crude methanol stream has water in an amount of less than about 10 wt. %, based on the total weight of the crude methanol stream.

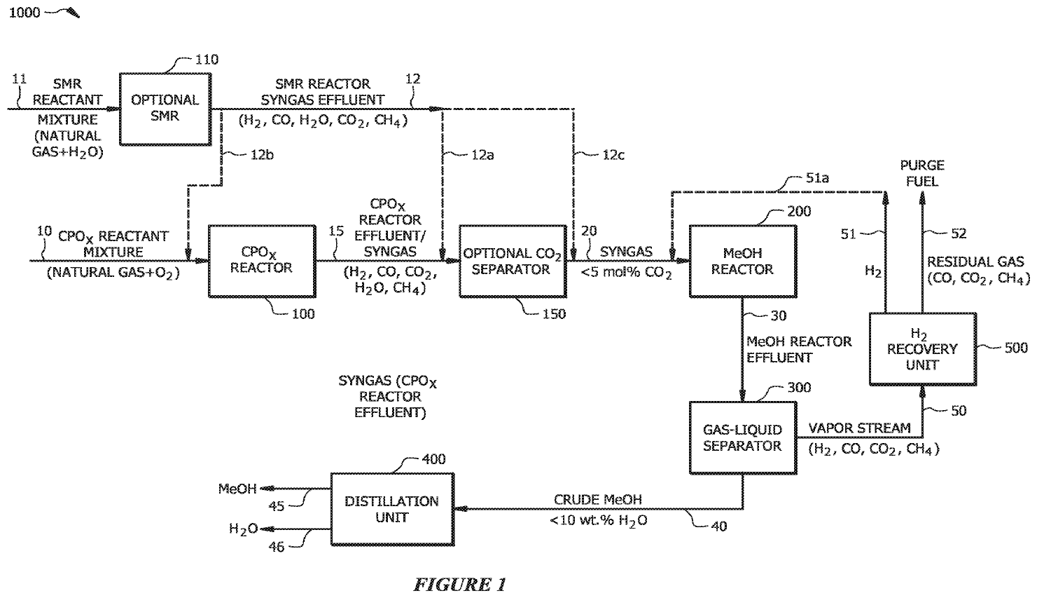

### TECHNICAL FIELD

The present disclosure relates to methods of producing methanol, more specifically methods of producing methanol from syngas produced by catalytic partial oxidation of hydrocarbons, such as methane.

### BACKGROUND

Synthesis gas (syngas) is a mixture comprising carbon monoxide (CO) and hydrogen (H₂), as well as small amounts of carbon dioxide (CO₂), water (H₂O), and unreacted methane (CH₄). Syngas is generally used as an intermediate in the production of methanol and ammonia, as well as an intermediate in creating synthetic petroleum to use as a lubricant or fuel.

Syngas is produced conventionally by steam reforming of natural gas (steam methane reforming or SMR), although other hydrocarbon sources can be used for syngas production, such as refinery off-gases, naphtha feedstocks, heavy hydrocarbons, coal, biomass, etc. SMR is an endothermic process and requires significant energy input to drive the reaction forward. Conventional endothermic technologies such as SMR produce syngas with a hydrogen content greater than the required content for methanol synthesis. Generally, SMR produces syngas with an M ratio ranging from 2.6 to 2.98, wherein the M ratio is a molar ratio defined as (H₂—CO₂)/(CO+CO₂).

In an autothermal reforming (ATR) process, a portion of the natural gas is burned as fuel to drive the conversion of natural gas to syngas resulting in relatively low hydrogen and high CO₂ concentrations. Conventional methanol production plants utilize a combined reforming (CR) technology that pairs SMR with autothermal reforming (ATR) to reduce the amount of hydrogen present in syngas. ATR produces a syngas with a hydrogen content lower than the required content for methanol synthesis. Generally, ATR produces syngas with an M ratio ranging from 1.7 to 1.84. In the CR technology, the natural gas feed volumetric flowrate to the SMR and the ATR can be adjusted to achieve an overall syngas M ratio of 2.0 to 2.06. Further, CR syngas has a hydrogen content greater than the required content for methanol synthesis. Furthermore, SMR is a highly endothermic process, and the endothermicity of the SMR technology requires burning fuel to drive the syngas synthesis. Consequently, the SMR technology reduces the energy efficiency of the methanol synthesis process.

Syngas can also be produced (non-commercially) by catalytic partial oxidation (CPO or CPOx) of natural gas. CPO processes employ partial oxidation of hydrocarbon feeds to syngas comprising CO and H₂. The CPO process is exothermic, thus eliminating the need for external heat supply. However, the composition of the produced syngas is not suitable for methanol synthesis, for example, owing to a reduced hydrogen content.

Further, in the conventional methanol synthesis processes, the purification (e.g., distillation) of the produced methanol is highly energy intensive. The purification (e.g., distillation) part of the methanol production process is primarily used to remove water from the crude methanol. The conventional methanol synthesis processes utilize multiple distillation trains for water removal and methanol purification, which renders the overall process energy intensive. Thus, there is an ongoing need for the development of methanol production processes that can control the composition of the produced crude methanol, for example by controlling the composition of the syngas used for producing the methanol.

### BRIEF DESCRIPTION OF THE DRAWINGS

For a detailed description of the preferred aspects of the disclosed methods, reference will now be made to the accompanying drawing in which:

The FIGURE displays a schematic of a system for a methanol production process.

### DETAILED DESCRIPTION

Disclosed herein are processes for producing methanol comprising (a) reacting, via a catalytic partial oxidation (CPO or CPOx) reaction, a CPO reactant mixture in a CPO reactor to produce syngas; wherein the CPO reactant mixture comprises hydrocarbons and oxygen; wherein the CPO reactor comprises a CPO catalyst; and wherein the syngas comprises hydrogen, carbon monoxide, carbon dioxide, water, and unreacted hydrocarbons; (b) introducing at least a portion of the syngas (e.g., subsequent to cooling and water removal from syngas; and/or subsequent to pressure and/or syngas temperature adjustment) to a methanol reactor to produce a methanol reactor effluent stream; wherein the methanol reactor effluent stream comprises methanol, water, hydrogen, carbon monoxide, carbon dioxide, and hydrocarbons; and (c) separating at least a portion of the methanol reactor effluent stream into a crude methanol stream and a vapor stream; wherein the crude methanol stream comprises methanol and water; wherein the vapor stream comprises hydrogen, carbon monoxide, carbon dioxide, and hydrocarbons; and wherein the crude methanol stream comprises water in an amount of less than about 10 wt. %, based on the total weight of the crude methanol stream. The hydrocarbons used for syngas production can comprise methane, natural gas, natural gas liquids, associated gas, well head gas, enriched gas, paraffins, shale gas, shale liquids, fluid catalytic cracking (FCC) off gas, refinery process gases, stack gases, fuel gas from fuel gas header, and the like, or combinations thereof.

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an aspect,” “another aspect,” “other aspects,” “some aspects,” and so forth, means that a particular element (e.g., feature, structure, property, and/or characteristic) described in connection with the aspect is included in at least an aspect described herein, and may or may not be present in other aspects. In addition, it is to be understood that the described element(s) can be combined in any suitable manner in the various aspects.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“—”) that is not between two letters or symbols is used to indicate a point of attachment for a substituent. For example, —CHO is attached through the carbon of the carbonyl group.

As used herein, the terms “Cₓ hydrocarbons” and “Cₓs” are interchangeable and refer to any hydrocarbon having x number of carbon atoms (C). For example, the terms “C₄ hydrocarbons” and “C₄s” both refer to any hydrocarbons having exactly 4 carbon atoms, such as n-butane, iso-butane, cyclobutane, 1-butene, 2-butene, isobutylene, butadiene, and the like, or combinations thereof.

As used herein, the term “Cₓ₊ hydrocarbons” refers to any hydrocarbon having equal to or greater than x carbon atoms (C). For example, the term “C₂₊ hydrocarbons” refers to any hydrocarbons having 2 or more carbon atoms, such as ethane, ethylene, C₃s, C₄s, C₅s, etc.

Referring to the FIGURE, a methanol production system 1000 is disclosed. The methanol production system 1000 generally comprises a catalytic partial oxidation (CPO or CPOx) reactor 100; an optional steam methane reforming (SMR) reactor 110; an optional carbon dioxide (CO₂) separator 150; a methanol reactor 200; a gas-liquid separator 300; a distillation unit 400; and a hydrogen (H₂) recovery unit 500. As will be appreciated by one of skill in the art, and with the help of this disclosure, methanol production system components shown in the FIGURE can be in fluid communication with each other (as represented by the connecting lines indicating a direction of fluid flow) through any suitable conduits (e.g., pipes, streams, etc.).

In an aspect, a process for producing methanol as disclosed herein can comprise a step of reacting, via a CPO reaction, a CPO reactant mixture 10 in the CPO reactor 100 to produce syngas (e.g., CPO reactor effluent 15); wherein the CPO reactant mixture 10 comprises hydrocarbons, oxygen, and optionally steam; wherein the CPO reactor 100 comprises a CPO catalyst; and wherein the syngas comprises hydrogen, carbon monoxide, carbon dioxide, water, and unreacted hydrocarbons.

Generally, the CPO reaction is based on partial combustion of fuels, such as various hydrocarbons, and in the case of methane, CPO can be represented by equation (1):

CH₄+1/2 O₂→CO+2 H₂ (1)

Without wishing to be limited by theory, side reactions can take place along with the CPO reaction depicted in equation (1); and such side reactions can produce carbon dioxide (CO₂) and water (H₂O), for example via hydrocarbon combustion, which is an exothermic reaction. As will be appreciated by one of skill in the art, and with the help of this disclosure, and without wishing to be limited by theory, the CPO reaction as represented by equation (1) can yield a syngas with a hydrogen to carbon monoxide (H₂/CO) molar ratio having the theoretical stoichiometric limit of 2.0. Without wishing to be limited by theory, the theoretical stoichiometric limit of 2.0 for the H₂/CO molar ratio means that the CPO reaction as represented by equation (1) yields 2 moles of H₂ for every 1 mole of CO, i.e., H₂/CO molar ratio of (2 moles H₂/1 mole CO)=2. As will be appreciated by one of skill in the art, and with the help of this disclosure, the theoretical stoichiometric limit of 2.0 for the H₂/CO molar ratio in a CPO reaction cannot be achieved practically because reactants (e.g., hydrocarbons, oxygen) as well as products (e.g., H₂, CO) undergo side reactions at the conditions used for the CPO reaction. As will be appreciated by one of skill in the art, and with the help of this disclosure, and without wishing to be limited by theory, in the presence of oxygen, CO and H₂ can be oxidized to CO₂ and H₂O, respectively. The relative amounts (e.g., composition) of CO, H₂, CO₂ and H₂O can be further altered by the equilibrium of the water-gas shift (WGS) reaction, which will be discussed in more detail later herein. The side reactions that can take place in the CPO reactor 100 can have a direct impact on the M ratio of the produced syngas, wherein the M ratio is a molar ratio defined as (H₂—CO₂)/(CO+CO₂). In the absence of any side reaction (theoretically), the CPO reaction as represented by equation (1) results in a syngas with an M ratio of 2.0. However, the presence of side reactions (practically) reduces H₂ and increases CO₂, thereby resulting in a syngas with an M ratio below 2.0.

Further, without wishing to be limited by theory, the CPO reaction as depicted in equation (1) is an exothermic heterogeneous catalytic reaction (i.e., a mildly exothermic reaction) and it occurs in a single reactor unit, such as the CPO reactor 100 (as opposed to more than one reactor unit as is the case in conventional processes for syngas production, such as steam methane reforming (SMR)—autothermal reforming (ATR) combinations). While it is possible to conduct partial oxidation of hydrocarbons as a homogeneous reaction, in the absence of a catalyst, homogeneous partial oxidation of hydrocarbons process entails excessive temperatures, long residence times, as well as excessive coke formation, which strongly reduce the controllability of the partial oxidation reaction, and may not produce syngas of the desired quality in a single reactor unit.

Furthermore, without wishing to be limited by theory, the CPO reaction is fairly resistant to chemical poisoning, and as such it allows for the use of a wide variety of hydrocarbon feedstocks, including some sulfur containing hydrocarbon feedstocks; which, in some cases, can enhance catalyst life-time and productivity. By contrast, conventional ATR processes have more restrictive feed requirements, for example in terms of content of impurities in the feed (e.g., feed to ATR is desulfurized), as well as hydrocarbon composition (e.g., ATR primarily uses CH₄-rich feed).

In an aspect, the hydrocarbons suitable for use in a CPO reaction as disclosed herein can include methane (CH₄), natural gas, natural gas liquids, associated gas, well head gas, enriched gas, paraffins, shale gas, shale liquids, fluid catalytic cracking (FCC) off gas, refinery process gases, stack gases, fuel gas from fuel gas header, and the like, or combinations thereof. The hydrocarbons can include any suitable hydrocarbons source, and can contain C₁-C₆ hydrocarbons, as well some heavier hydrocarbons.

In an aspect, the CPO reactant mixture 10 can comprise natural gas. Generally, natural gas is composed primarily of methane, but can also contain ethane, propane and heavier hydrocarbons (e.g., iso-butane, n-butane, iso-pentane, n-pentane, hexanes, etc.), as well as very small quantities of nitrogen, oxygen, carbon dioxide, sulfur compounds, and/or water. The natural gas can be provided from a variety of sources including, but not limited to, gas fields, oil fields, coal fields, fracking of shale fields, biomass, landfill gas, and the like, or combinations thereof. In some aspects, the CPO reactant mixture 10 can comprise CH₄ and O₂.

The natural gas can comprise any suitable amount of methane. In some aspects, the natural gas can comprise biogas. For example, the natural gas can comprise from about 45 mol % to about 80 mol % methane, from about 20 mol % to about 55 mol % carbon dioxide, and less than about 15 mol % nitrogen.

In an aspect, natural gas can comprise CH₄ in an amount of equal to or greater than about 45 mol %, alternatively equal to or greater than about 50 mol %, alternatively equal to or greater than about 55 mol %, alternatively equal to or greater than about 60 mol %, alternatively equal to or greater than about 65 mol %, alternatively equal to or greater than about 70 mol %, alternatively equal to or greater than about 75 mol %, alternatively equal to or greater than about 80 mol %, alternatively equal to or greater than about 82 mol %, alternatively equal to or greater than about 84 mol %, alternatively equal to or greater than about 86 mol %, alternatively equal to or greater than about 88 mol %, alternatively equal to or greater than about 90 mol %, alternatively equal to or greater than about 91 mol %, alternatively equal to or greater than about 92 mol %, alternatively equal to or greater than about 93 mol %, alternatively equal to or greater than about 94 mol %, alternatively equal to or greater than about 95 mol %, alternatively equal to or greater than about 96 mol %, alternatively equal to or greater than about 97 mol %, alternatively equal to or greater than about 98 mol %, or alternatively equal to or greater than about 99 mol %.

In some aspects, the hydrocarbons suitable for use in a CPO reaction as disclosed herein can comprise C₁-C₆ hydrocarbons, nitrogen (e.g., from about 0.1 mol % to about 15 mol %, alternatively from about 0.5 mol % to about 11 mol %, alternatively from about 1 mol % to about 7.5 mol %, or alternatively from about 1.3 mol % to about 5.5 mol %), and carbon dioxide (e.g., from about 0.1 mol % to about 2 mol %, alternatively from about 0.2 mol % to about 1 mol %, or alternatively from about 0.3 mol % to about 0.6 mol %). For example, the hydrocarbons suitable for use in a CPO reaction as disclosed herein can comprise Cᵢ hydrocarbon (about 89 mol % to about 92 mol %); C₂ hydrocarbons (about 2.5 mol % to about 4 mol %); C₃ hydrocarbons (about 0.5 mol % to about 1.4 mol %); C₄ hydrocarbons (about 0.5 mol % to about 0.2 mol %); C₅ hydrocarbons (about 0.06 mol %); and C₆ hydrocarbons (about 0.02 mol %); and optionally nitrogen (about 0.1 mol % to about 15 mol %), carbon dioxide (about 0.1 mol % to about 2 mol %), or both nitrogen (about 0.1 mol % to about 15 mol %) and carbon dioxide (about 0.1 mol % to about 2 mol %).

The oxygen used in the CPO reactant mixture10 can comprise 100% oxygen (substantially pure O₂), oxygen gas (which may be obtained via a membrane separation process), technical oxygen (which may contain some air), air, oxygen enriched air, oxygen-containing gaseous compounds (e.g., NO), oxygen-containing mixtures (e.g., O₂/CO₂, O₂/H₂O, O₂/H₂O₂/H₂O), oxy radical generators (e.g., CH₃OH, CH₂O), hydroxyl radical generators, and the like, or combinations thereof.

In an aspect, the CPO reactant mixture 10 can be characterized by a carbon to oxygen (C/O) molar ratio of less than about 3:1, alternatively less than about 2.6:1, alternatively less than about 2.4:1, alternatively less than about 2.2:1, alternatively less than about 2:1, alternatively less than about 1.9:1, alternatively equal to or greater than about 2:1, alternatively equal to or greater than about 2.2:1, alternatively equal to or greater than about 2.4:1, alternatively equal to or greater than about 2.6:1, alternatively from about 0.5:1 to about 3:1, alternatively from about 0.7:1 to about 2.5:1, alternatively from about 0.9:1 to about 2.2:1, alternatively from about 1:1 to about 2:1, alternatively from about 1.1:1 to about 1.9:1, alternatively from about 2:1 to about 3:1, alternatively from about 2.2:1 to about 3:1, alternatively from about 2.4:1 to about 3:1, or alternatively from about 2.6:1 to about 3:1, wherein the C/O molar ratio refers to the total moles of carbon (C) of hydrocarbons in the reactant mixture divided by the total moles of oxygen (O₂) in the reactant mixture.

For example, when the only source of carbon in the CPO reactant mixture 10 is CH₄, the CH₄/O₂ molar ratio is the same as the C/O molar ratio. As another example, when the CPO reactant mixture 10 contains other carbon sources besides CH₄, such as ethane (C₂H₆), propane (C₃H₈), butanes (GPO, etc., the C/O molar ratio accounts for the moles of carbon in each compound (e.g., 2 moles of C in 1 mole of C₂H₆, 3 moles of C in 1 mole of C₃H₈, 4 moles of C in 1 mole of C₄H₁₀, etc.). As will be appreciated by one of skill in the art, and with the help of this disclosure, the C/O molar ratio in the CPO reactant mixture 10 can be adjusted along with other reactor process parameters (e.g., temperature, pressure, flow velocity, etc.) to provide for a syngas with a desired composition (e.g., a syngas with a desired CO₂ content, such as a syngas with a CO₂ content of from about 0.1 mol % to about 5 mol %). The C/O molar ratio in the CPO reactant mixture can be adjusted to provide for a decreased amount of unconverted hydrocarbons in the syngas. The C/O molar ratio in the CPO reactant mixture 10 can be adjusted based on the CPO effluent temperature in order to decrease (e.g., minimize) the unconverted hydrocarbons content of the produced syngas. As will be appreciated by one of skill in the art, and with the help of this disclosure, the C/O molar ratio can be adjusted along with other reactor process parameters (e.g., temperature, pressure, flow velocity, etc.) to provide for a syngas with a desired composition (e.g., a syngas with a desired CO₂ content, such as a syngas with a CO₂ content of from about 0.1 mol % to about 5 mol %).

The CPO reaction is an exothermic reaction (e.g., heterogeneous catalytic reaction; exothermic heterogeneous catalytic reaction) that is generally conducted in the presence of a CPO catalyst comprising a catalytically active metal, i.e., a metal active for catalyzing the CPO reaction. The catalytically active metal can comprise a noble metal (e.g., Pt, Rh, Ir, Pd, Ru, Ag, and the like, or combinations thereof); a non-noble metal (e.g., Ni, Co, V, Mo, P, Fe, Cu, and the like, or combinations thereof); rare earth elements (e.g., La, Ce, Nd, Eu, and the like, or combinations thereof); oxides thereof; and the like; or combinations thereof. Generally, a noble metal is a metal that resists corrosion and oxidation in a water-containing environment. As will be appreciated by one of skill in the art, and with the help of this disclosure, the components of the CPO catalyst (e.g., metals such as noble metals, non-noble metals, rare earth elements) can be either phase segregated or combined within the same phase.

In an aspect, the CPO catalysts suitable for use in the present disclosure can be supported catalysts and/or unsupported catalysts. In some aspects, the supported catalysts can comprise a support, wherein the support can be catalytically active (e.g., the support can catalyze a CPO reaction). For example, the catalytically active support can comprise a metal gauze or wire mesh (e.g., Pt gauze or wire mesh); a catalytically active metal monolithic catalyst; etc. In other aspects, the supported catalysts can comprise a support, wherein the support can be catalytically inactive (e.g., the support cannot catalyze a CPO reaction), such as SiO₂; silicon carbide (SiC); alumina; a catalytically inactive monolithic support; etc. In yet other aspects, the supported catalysts can comprise a catalytically active support and a catalytically inactive support.

In some aspects, a CPO catalyst can be wash coated onto a support, wherein the support can be catalytically active or inactive, and wherein the support can be a monolith, a foam, an irregular catalyst particle, etc.

In some aspects, the CPO catalyst can be a monolith, a foam, a powder, a particle, etc. Nonlimiting examples of CPO catalyst particle shapes suitable for use in the present disclosure include cylindrical, discoidal, spherical, tabular, ellipsoidal, equant, irregular, cubic, acicular, and the like, or combinations thereof.

In some aspects, the support comprises an inorganic oxide, alpha, beta or theta alumina (A1₂0₃), activated Al₂O₃, silicon dioxide (SiO₂), titanium dioxide (TiO₂), magnesium oxide (MgO), zirconium oxide (ZrO₂), lanthanum (III) oxide (La₂O₃), yttrium (III) oxide (Y₂O₃), cerium (IV) oxide (CeO₂), zeolites, ZSM-5, perovskite oxides, hydrotalcite oxides, and the like, or combinations thereof.

CPO processes, CPO reactors, CPO catalysts, and CPO catalyst bed configurations suitable for use in the present disclosure are described in more detail in U.S. Provisional Patent Application No. 62/522,910 filed June 21, 2017 (International Application No. PCT/IB2018/054475 filed Jun. 18, 2018) and entitled “Improved Reactor Designs for Heterogeneous Catalytic Reactions;” and U.S. Provisional Patent Application No. 62/521,831 filed Jun. 19, 2017 (International Application No. PCT/IB2018/054470 filed Jun. 18, 2018) and entitled “An Improved Process for Syngas Production for Petrochemical Applications;” each of which is incorporated by reference herein in its entirety.

In an aspect, a CPO reactor suitable for use in the present disclosure (e.g., CPO reactor 100) can comprise a tubular reactor, a continuous flow reactor, an isothermal reactor, an adiabatic reactor, a fixed bed reactor, a fluidized bed reactor, a bubbling bed reactor, a circulating bed reactor, an ebullated bed reactor, a rotary kiln reactor, and the like, or combinations thereof.

In some aspects, the CPO reactor 100 can be characterized by at least one CPO operational parameter selected from the group consisting of a CPO reactor temperature (e.g., CPO catalyst bed temperature); CPO feed temperature (e.g., CPO reactant mixture temperature); target CPO effluent temperature; a CPO pressure (e.g., CPO reactor pressure); a CPO contact time (e.g., CPO reactor contact time); a C/O molar ratio in the CPO reactant mixture; a steam to carbon (S/C) molar ratio in the CPO reactant mixture, wherein the S/C molar ratio refers to the total moles of water (H₂O) in the reactant mixture divided by the total moles of carbon (C) of hydrocarbons in the reactant mixture; and combinations thereof. For purposes of the disclosure herein, the CPO effluent temperature is the temperature of the syngas (e.g., syngas effluent) measured at the point where the syngas exits the CPO reactor (CPO reactor 100), e.g., a temperature of the syngas measured at a CPO reactor outlet, a temperature of the syngas effluent, a temperature of the exit syngas effluent. For purposes of the disclosure herein, the CPO effluent temperature (e.g., target CPO effluent temperature) is considered an operational parameter. As will be appreciated by one of skill in the art, and with the help of this disclosure, the choice of operational parameters for the CPO reactor such as CPO feed temperature; CPO pressure; CPO contact time; C/O molar ratio in the CPO reactant mixture; S/C molar ratio in the CPO reactant mixture; etc. determines the temperature of CPO reactor effluent (e.g., syngas), as well as the composition of the CPO reactor effluent (e.g., syngas). Further, and as will be appreciated by one of skill in the art, and with the help of this disclosure, monitoring the CPO effluent temperature can provide feedback for changing other operational parameters (e.g., CPO feed temperature; CPO pressure; CPO contact time; C/O molar ratio in the CPO reactant mixture; S/C molar ratio in the CPO reactant mixture; etc.) as necessary for the CPO effluent temperature to match the target CPO effluent temperature. Furthermore, and as will be appreciated by one of skill in the art, and with the help of this disclosure, the target CPO effluent temperature is the desired CPO effluent temperature, and the CPO effluent temperature (e.g., measured CPO effluent temperature, actual CPO effluent temperature) may or may not coincide with the target CPO effluent temperature. In aspects where the CPO effluent temperature is different from the target CPO effluent temperature, one or more CPO operational parameters (e.g., CPO feed temperature; CPO pressure; CPO contact time; C/O molar ratio in the CPO reactant mixture; S/C molar ratio in the CPO reactant mixture; etc.) can be adjusted (e.g., modified) in order for the CPO effluent temperature to match (e.g., be the same with, coincide with) the target CPO effluent temperature. The CPO reactor 100 can be operated under any suitable operational parameters that can provide for a syngas with a desired composition (e.g., a syngas with a desired CO₂ content, such as a syngas with a CO₂ content of from about 0.1 mol % to about 5 mol %).

The CPO reactor 100 can be characterized by a CPO feed temperature of from about 25° C. to about 600° C., alternatively from about 25° C. to about 500° C., alternatively from about 25° C. to about 400° C., alternatively from about 50° C. to about 400° C., or alternatively from about 100° C. to about 400° C. In aspects where the CPO reactant mixture comprises steam, the CPO feed temperature can be as high as about 600° C., alternatively about 575° C., alternatively about 550° C., or alternatively about 525° C. In aspects where the CPO reactant mixture does not comprise steam, the CPO feed temperature can be as high as about 450° C., alternatively about 425° C., alternatively about 400° C., or alternatively about 375° C.

The CPO reactor 100 can be characterized by a CPO effluent temperature (e.g., target CPO effluent temperature) of equal to or greater than about 300° C., alternatively equal to or greater than about 600° C., alternatively equal to or greater than about 700° C., alternatively equal to or greater than about 750° C., alternatively equal to or greater than about 800° C., alternatively equal to or greater than about 850° C., alternatively from about 300° C. to about 1,600° C., alternatively from about 600° C. to about 1,400° C., alternatively from about 600° C. to about 1,300° C., alternatively from about 700° C. to about 1,200° C., alternatively from about 750° C. to about 1,150° C., alternatively from about 800° C. to about 1,125° C., or alternatively from about 850° C. to about 1,100° C.

In an aspect, the CPO reactor 100 can be characterized by any suitable reactor temperature and/or catalyst bed temperature. For example, the CPO reactor 100 can be characterized by a reactor temperature and/or catalyst bed temperature of equal to or greater than about 300° C., alternatively equal to or greater than about 600° C., alternatively equal to or greater than about 700° C., alternatively equal to or greater than about 750° C., alternatively equal to or greater than about 800° C., alternatively equal to or greater than about 850° C., alternatively from about 300° C. to about 1,600° C., alternatively from about 600° C. to about 1,400° C., alternatively from about 600° C. to about 1,300° C., alternatively from about 700° C. to about 1,200° C., alternatively from about 750° C. to about 1,150° C., alternatively from about 800° C. to about 1,125° C., or alternatively from about 850° C. to about 1,100° C.

The CPO reactor 100 can be operated under any suitable temperature profile that can provide for a syngas with a desired composition (e.g., a syngas with a desired CO₂ content; such as a syngas with a CO₂ content of less than about 5 mol %, alternatively less than about 4 mol %, alternatively less than about 3 mol %, alternatively less than about 2 mol %, alternatively less than about 1 mol %, alternatively from about 0.1 mol % to about 5 mol %, alternatively from about 0.25 mol % to about 4 mol %, or alternatively from about 0.5 mol % to about 3 mol %). The CPO reactor 100 can be operated under adiabatic conditions, non-adiabatic conditions, isothermal conditions, near-isothermal conditions, etc. For purposes of the disclosure herein, the term “non-adiabatic conditions” refers to process conditions wherein a reactor is subjected to external heat exchange or transfer (e.g., the reactor is heated; or the reactor is cooled), which can be direct heat exchange and/or indirect heat exchange. As will be appreciated by one of skill in the art, and with the help of this disclosure, the terms “direct heat exchange” and “indirect heat exchange” are known to one of skill in the art. By contrast, the term “adiabatic conditions” refers to process conditions wherein a reactor is not subjected to external heat exchange (e.g., the reactor is not heated; or the reactor is not cooled). Generally, external heat exchange implies an external heat exchange system (e.g., a cooling system; a heating system) that requires energy input and/or output. As will be appreciated by one of skill in the art, and with the help of this disclosure, external heat transfer can also result from heat loss from the catalyst bed (or reactor) owing to radiation heat transfer, conduction heat transfer, convection heat transfer, and the like, or combinations thereof. For example, the catalyst bed can participate in heat exchange with the external environment, and/or with reactor zones upstream and/or downstream of the catalyst bed.

For purposes of the disclosure herein, the term “isothermal conditions” refers to process conditions (e.g., CPO operational parameters) that allow for a substantially constant temperature of the reactor and/or catalyst bed (e.g., isothermal temperature) that can be defined as a temperature that varies by less than about ±10° C., alternatively less than about ±9° C., alternatively less than about ±8° C., alternatively less than about ±7° C., alternatively less than about ±6° C., alternatively less than about ±5° C., alternatively less than about ±4° C., alternatively less than about ±3° C., alternatively less than about ±2° C., or alternatively less than about ±1° C. across the reactor and/or catalyst bed, respectively.

Further, for purposes of the disclosure herein, the term “isothermal conditions” refers to process conditions (e.g., CPO operational parameters) effective for providing for a syngas with a desired composition (e.g., a desired H₂/CO molar ratio; a desired CO₂ content; etc.), wherein the isothermal conditions comprise a temperature variation of less than about ±10° C. across the reactor and/or catalyst bed.

The CPO reactor 100 can be operated under any suitable operational parameters that can provide for isothermal conditions.

For purposes of the disclosure herein, the term “near-isothermal conditions” refers to process conditions (e.g., CPO operational parameters) that allow for a fairly constant temperature of the reactor and/or catalyst bed (e.g., near-isothermal temperature), which can be defined as a temperature that varies by less than about ±100° C., alternatively less than about ±90° C., alternatively less than about ±80° C., alternatively less than about ±70° C., alternatively less than about ±60° C., alternatively less than about ±50° C., alternatively less than about ±40° C., alternatively less than about ±30° C., alternatively less than about ±20° C., alternatively less than about ±10° C., alternatively less than about ±9° C., alternatively less than about ±8° C., alternatively less than about ±7° C., alternatively less than about ±6° C., alternatively less than about ±5° C., alternatively less than about ±4° C., alternatively less than about ±3° C., alternatively less than about ±2° C., or alternatively less than about ±1° C. across the reactor and/or catalyst bed, respectively. In some aspects, near-isothermal conditions allow for a temperature variation of less than about ±50° C., alternatively less than about ±25° C., or alternatively less than about ±10° C. across the reactor and/or catalyst bed. Further, for purposes of the disclosure herein, the term “near-isothermal conditions” is understood to include “isothermal” conditions.

Furthermore, for purposes of the disclosure herein, the term “near-isothermal conditions” refers to process conditions (e.g., CPO operational parameters) effective for providing for a syngas with a desired composition (e.g., a desired H₂/CO molar ratio; a desired CO₂ content; etc.), wherein the near-isothermal conditions comprise a temperature variation of less than about ±100° C. across the reactor and/or catalyst bed.

In an aspect, a process as disclosed herein can comprise conducting the CPO reaction under near-isothermal conditions to produce syngas, wherein the near-isothermal conditions comprise a temperature variation of less than about ±100° C. across the reactor and/or catalyst bed.

The CPO reactor 100 can be operated under any suitable operational parameters that can provide for near-isothermal conditions.

The CPO reactor 100 can be characterized by a CPO pressure (e.g., reactor pressure measured at the reactor exit or outlet) of equal to or greater than about 1 barg, alternatively equal to or greater than about 10 barg, alternatively equal to or greater than about 20 barg, alternatively equal to or greater than about 25 barg, alternatively equal to or greater than about 30 barg, alternatively equal to or greater than about 35 barg, alternatively equal to or greater than about 40 barg, alternatively equal to or greater than about 50 barg, alternatively less than about 30 barg, alternatively less than about 25 barg, alternatively less than about 20 barg, alternatively less than about 10 barg, from about 1 barg to about 90 barg, alternatively from about 1 barg to about 40 barg, alternatively from about 1 barg to about 30 barg, alternatively from about 1 barg to about 25 barg, alternatively from about 1 barg to about 20 barg, alternatively from about 1 barg to about 10 barg, alternatively from about 20 barg to about 90 barg, alternatively from about 25 barg to about 85 barg, or alternatively from about 30 barg to about 80 barg.

The CPO reactor 100 can be characterized by a CPO contact time of from about 0.001 milliseconds (ms) to about 5 seconds (s), alternatively from about 0.001 ms to about 1 s, alternatively from about 0.001 ms to about 100 ms, alternatively from about 0.001 ms to about 10 ms, alternatively from about 0.001 ms to about 5 ms, or alternatively from about 0.01 ms to about 1.2 ms. Generally, the contact time of a reactor comprising a catalyst refers to the average amount of time that a compound (e.g., a molecule of that compound) spends in contact with the catalyst (e.g., within the catalyst bed), e.g., the average amount of time that it takes for a compound (e.g., a molecule of that compound) to travel through the catalyst bed. For purposes of the disclosure herein the contact time of less than about 5 ms can be referred to as “millisecond regime” (MSR); and a CPO process or CPO reaction as disclosed herein characterized by a contact time of less than about 5 ms can be referred to as “millisecond regime”-CPO (MSR-CPO) process or reaction, respectively.

In some aspects, the CPO reactor 100 can be characterized by a contact time of from about 0.001 ms to about 5 ms, or alternatively from about 0.01 ms to about 1.2 ms.

All of the CPO operational parameters disclosed herein are applicable throughout all of the embodiments disclosed herein, unless otherwise specified. As will be appreciated by one of skill in the art, and with the help of this disclosure, each CPO operational parameter can be adjusted to provide for a desired syngas quality, such as a syngas with a desired composition (e.g., a syngas with a desired CO₂ content, such as a syngas with a CO₂ content of from about 0.1 mol % to about 5 mol %). For example, the CPO operational parameters can be adjusted to provide for a decreased CO₂ content of the syngas. As another example, the CPO operational parameters can be adjusted to provide for an increased H₂ content of the syngas. As yet another example, the CPO operational parameters can be adjusted to provide for a decreased unreacted hydrocarbons (e.g., unreacted CH₄) content of the syngas.

In an aspect, a CPO reactor effluent 15 can be recovered from the CPO reactor 100, wherein the CPO reactor effluent 15 comprises hydrogen, carbon monoxide, water, carbon dioxide, and unreacted hydrocarbons.

In some aspects, the CPO reactor effluent 15 (e.g., subsequent to cooling and water removal from syngas; and/or subsequent to pressure and/or syngas temperature adjustment) can be used as syngas 20 in a downstream process (e.g., methanol production) without further processing to enrich the hydrogen content and/or decrease the CO₂ content of the CPO reactor effluent 15. In such aspects, CPO reactor effluent 15 is the same stream as syngas 20, wherein the H₂/CO molar ratio of the CPO reactor effluent 15 is the same as the H₂/CO molar ratio of the syngas 20. The CPO reactor effluent 15 and/or syngas 20 as disclosed herein can be characterized by a H₂/CO molar ratio of greater than about 1.7, alternatively greater than about 1.8, alternatively greater than about 1.9, alternatively greater than about 2.0, alternatively greater than about 2.2, alternatively greater than about 2.5, alternatively greater than about 2.7, or alternatively greater than about 3.0. In some aspects, the CPO reactor effluent 15 and/or syngas 20 as disclosed herein can be characterized by a H₂/CO molar ratio of from about 1.7 to about 2.3, alternatively from about 1.8 to about 2.2, or alternatively from about 1.9 to about 2.1.

In other aspects, the CPO reactor effluent 15 can be further processed to produce the syngas 20, wherein the syngas 20 can be further used for methanol production. The CPO reactor effluent 15 can be processed to enrich its hydrogen content. In such aspects, the H₂/CO molar ratio of the syngas 20 is greater than the H₂/CO molar ratio of the CPO reactor effluent 15.

As will be appreciated by one of skill in the art, and with the help of this disclosure, although the syngas 20 can be characterized by a H₂/CO molar ratio of greater than about 1.8, which can be appropriate for methanol synthesis, the syngas 20 can be processed to further decrease its CO₂ content, to provide for a syngas with a desired composition (e.g., a syngas with a desired CO₂ content, such as a syngas with a CO₂ content of from about 0.1 mol % to about 5 mol %).

Further, as will be appreciated by one of skill in the art, and with the help of this disclosure, the CPO reactor effluent 15 and/or syngas 20 can be subjected to minimal processing, such as the recovery of unreacted hydrocarbons, diluent, water, etc., without substantially changing the H₂/CO molar ratio of the CPO reactor effluent 15 and/or syngas 20, respectively. For example, water can be condensed and separated from the CPO reactor effluent 15 and/or syngas 20, e.g., in a condenser.

In an aspect, a process for producing methanol as disclosed herein can further comprise (i) recovering at least a portion of the unreacted hydrocarbons from the CPO reactor effluent 15 and/or syngas 20 to yield recovered hydrocarbons, and (ii) recycling at least a portion of the recovered hydrocarbons to the CPO reactor 100. As will be appreciated by one of skill in the art, and with the help of this disclosure, although fairly high conversions can be achieved in CPO processes (e.g., conversions of equal to or greater than about 90%), the unconverted hydrocarbons could be recovered and recycled back to the CPO reactor 100.

The CPO reactor 100 can be operated under any suitable operational parameters that can provide for a syngas with a desired composition (e.g., a syngas with a desired CO₂ content, such as a syngas with a CO₂ content of from about 0.1 mol % to about 5 mol %); for example, the CPO reactor 100 can be operated at relatively low pressure, and optionally at relatively low C/O molar ratio in the CPO reactant mixture 10. Without wishing to be limited by theory, for a given CPO effluent temperature (e.g., target CPO effluent temperature) and a given C/O molar ratio in the CPO reactant mixture, the H₂/CO molar ratio of the produced syngas increases with decreasing the pressure. Further, without wishing to be limited by theory, and according to Le Chatelier's Principle, the equilibrium of the reforming reaction represented by equation (3) will be shifted towards producing H₂ and CO with decreasing the pressure: the reforming reaction goes from 2 moles reactants (CH₄ and H₂O) to 4 moles of products (H₂ and CO), and a decrease in pressure will favor the equilibrium of the reaction to be shifted towards the production of H₂ and CO. The reforming reaction represented by equation (3) can lead to a syngas having a H₂/CO molar ratio of 3, which is greater than the H₂/CO molar ratio of 2 for the syngas produced according to the CPO reaction as represented by equation (1).

In an aspect, the CPO reactor 100 can be operated at a CPO pressure of less than about 30 barg, alternatively less than about 25 barg, alternatively less than about 20 barg, alternatively less than about 10 barg, alternatively from about 1 barg to about 30 barg, alternatively from about 1 barg to about 25 barg, alternatively from about 1 barg to about 20 barg, or alternatively from about 1 barg to about 10 barg. In such aspect, the CPO reactor 100 can be operated at (i) a CPO effluent temperature (e.g., target CPO effluent temperature) of equal to or greater than about 750° C., alternatively equal to or greater than about 800° C., alternatively equal to or greater than about 850° C., alternatively from about 750° C. to about 1,150° C., alternatively from about 800° C. to about 1,125° C., or alternatively from about 850° C. to about 1,100° C.; and/or (ii) a C/O molar ratio in the CPO reactant mixture 10 of less than about 2.2:1, alternatively less than about 2:1, alternatively less than about 1.9:1, alternatively from about 0.9:1 to about 2.2:1, alternatively from about 1:1 to about 2:1, or alternatively from about 1.1:1 to about 1.9:1.

In some aspects, the CPO reactor 100 can be operated at a CPO pressure of less than about 30 barg, at a CPO effluent temperature (e.g., target CPO effluent temperature) of equal to or greater than about 750° C., at a C/O molar ratio in the CPO reactant mixture 10 of less than about 2.2:1, and at a S/C molar ratio in the CPO reactant mixture of from about 0.2:1 to about 0.8:1.

The CPO reactor can be operated under any suitable operational parameters that can provide for a syngas with a desired composition (e.g., a syngas with a desired CO₂ content, such as a syngas with a CO₂ content of from about 0.1 mol % to about 5 mol %); for example, the CPO reactor 100 can be operated at a relatively high C/O molar ratio in the CPO reactant mixture 10, and optionally at relatively low pressure.

When excess hydrocarbons (e.g., methane) are present, a portion of hydrocarbons can undergo a thermal decomposition reaction, for example as represented by equation (2):

CH₄→C+2 H₂ (2)

The decomposition reaction of hydrocarbons, such as methane, is facilitated by elevated temperatures, and increases the hydrogen content in the CPO reactor effluent 15 and/or syngas 20. As will be appreciated by one of skill in the art, and with the help of this disclosure, and without wishing to be limited by theory, while the percentage of hydrocarbons in the CPO reactant mixture 10 that undergoes a decomposition reaction (e.g., a decomposition reaction as represented by equation (2)) increases with increasing the C/O molar ratio in the CPO reactant mixture 10, a portion of hydrocarbons can undergo a decomposition reaction to carbon (C) and H₂ even at relatively low C/O molar ratios in the CPO reactant mixture 10 (e.g., a C/O molar ratio in the CPO reactant mixture 10 of less than about 2:1).

In an aspect, the CPO reactor 100 can be operated at a C/O molar ratio in the CPO reactant mixture 10 of equal to or greater than about 2:1, alternatively equal to or greater than about 2.2:1, alternatively equal to or greater than about 2.4:1, alternatively equal to or greater than about 2.6:1, alternatively from about 2:1 to about 3:1, alternatively from about 2.2:1 to about 3:1, alternatively from about 2.4:1 to about 3:1, or alternatively from about 2.6:1 to about 3:1. In such aspect, the CPO reactor 100 can be operated at (i) a CPO pressure of less than about 30 barg, alternatively less than about 25 barg, alternatively less than about 20 barg, alternatively less than about 10 barg, alternatively from about 1 barg to about 30 barg, alternatively from about 1 barg to about 25 barg, alternatively from about 1 barg to about 20 barg, or alternatively from about 1 barg to about 10 barg; and/or (ii) a CPO effluent temperature (e.g., target CPO effluent temperature) of equal to or greater than about 750° C., alternatively equal to or greater than about 800° C., alternatively equal to or greater than about 850° C., alternatively from about 750° C. to about 1,150° C., alternatively from about 800° C. to about 1,125° C., or alternatively from about 850° C. to about 1,100° C.

In some aspects, the CPO reactor 100 can be operated at a CPO pressure of less than about 30 barg, at a CPO effluent temperature (e.g., target CPO effluent temperature) of equal to or greater than about 750° C., and at a C/O molar ratio in the CPO reactant mixture 10 of equal to or greater than about 2:1.

In an aspect, the CPO reactant mixture 10 can further comprise a diluent, such as water and/or steam. The CPO reactor 100 can be operated under any suitable operational parameters that can provide for a syngas with a desired composition (e.g., a syngas with a desired CO₂ content, such as a syngas with a CO₂ content of from about 0.1 mol % to about 5 mol %); for example, the CPO reactor 100 can be operated with introducing water and/or steam to the CPO reactor 100.

Generally, a diluent is inert with respect to the CPO reaction, e.g., the diluent does not participate in the CPO reaction (e.g., a CPO reaction as represented by equation (1)). However, and as will be appreciated by one of skill in the art, and with the help of this disclosure, some diluents (e.g., water, steam, etc.) might undergo chemical reactions other than the CPO reaction within the CPO reactor 100, and can change the composition of the resulting syngas (e.g., CPO reactor effluent 15 and/or syngas 20). As will be appreciated by one of skill in the art, and with the help of this disclosure, water and/or steam can be used to vary the composition of the resulting syngas. Steam can react with methane, for example as represented by equation (3):

CH₄+H₂O [symbol] CO+3H₂ (3)

In an aspect, a diluent comprising water and/or steam can increase a hydrogen content of the resulting syngas (e.g., CPO reactor effluent 15 and/or syngas 20). For example, in aspects where the CPO reactant mixture 10 comprises water and/or steam diluent, the resulting syngas (e.g., CPO reactor effluent 15 and/or syngas 20) can be characterized by a hydrogen to carbon monoxide molar ratio that is increased when compared to a hydrogen to carbon monoxide molar ratio of a syngas produced by an otherwise similar process conducted with a reactant mixture comprising hydrocarbons and oxygen without the water and/or steam diluent.

Further, in the presence of water and/or steam in the CPO reactor 100, carbon monoxide can react with the water and/or steam to form carbon dioxide and hydrogen via a water-gas shift (WGS) reaction, for example as represented by equation (4):

CO+H₂O [symbol] CO₂+H₂ (4)

While the WGS reaction can increase the H₂/CO molar ratio of the syngas produced by the CPO reactor 200, it also produces CO₂.

When carbon is present in the reactor (e.g., coke; C produced as a result of a decomposition reaction as represented by equation (2)), water and/or steam diluent can react with the carbon and generate additional CO and H₂, for example as represented by equation (5):

C+H₂O [symbol] CO+H₂ (5)

Further, since oxygen is present in the CPO reactant mixture 10, the carbon present in the reactor (e.g., coke; C produced as a result of a decomposition reaction as represented by equation (2)) can also react with oxygen, for example as represented by equation (6):

C+O₂→CO₂ (6)

Furthermore, CO₂ can react with carbon (e.g., coke; C produced as a result of a decomposition reaction as represented by equation (2)), for example as represented by equation (7):

C+CO₂ [symbol] 2 CO (7)

thereby decreasing the amount of CO₂ in the resulting syngas (e.g., CPO reactor effluent 15 and/or syngas 20).

Furthermore, CO₂ can react with methane in a dry reforming reaction, for example as represented by equation (8):

CH₄+CO₂ [symbol] 2CO+2H₂ (8)

thereby decreasing the amount of CO₂ in the resulting syngas (e.g., CPO reactor effluent 15 and/or syngas 20).

In an aspect, the CPO reactor 100 can be operated at a steam to carbon (S/C) molar ratio in the CPO reactant mixture of less than about 2.4:1, alternatively less than about 2:1, alternatively less than about 1.5:1, alternatively less than about 1:1, alternatively less than about 0.8:1, alternatively less than about 0.5:1, alternatively from about 0.01:1 to less than about 2.4:1, alternatively from about 0.05:1 to about 2:1, alternatively from about 0.1:1 to about 1.5:1, alternatively from about 0.15:1 to about 1:1, or alternatively from about 0.2:1 to about 0.8:1, wherein the S/C molar ratio refers to the total moles of water (H₂O) in the reactant mixture divided by the total moles of carbon (C) of hydrocarbons in the reactant mixture. As will be appreciated by one of skill in the art, and with the help of this disclosure, the steam that is introduced to the reactor for use as a diluent in a CPO reaction as disclosed herein is present in significantly smaller amounts than the amounts of steam utilized in steam reforming (e.g., SMR) processes, and as such, a process for producing syngas as disclosed herein can yield a syngas with lower amounts of hydrogen when compared to the amounts of hydrogen in a syngas produced by steam reforming.

The S/C molar ratio in the CPO reactant mixture 10 can be adjusted based on the desired CPO effluent temperature (e.g., target CPO effluent temperature) in order to increase (e.g., maximize) the H₂ content of the produced syngas. As will be appreciated by one of skill in the art, and with the help of this disclosure, the reaction (3) that consumes steam in the CPO reactor 100 is preferable over the water-gas shift (WGS) reaction (4) in the CPO reactor 100, as reaction (3) allows for increasing the H₂ content of the produced syngas, as well as the M ratio of the produced syngas, wherein the M ratio is a molar ratio defined as (H₂—CO₂)/(CO+CO₂).

In an aspect, the amount of methane that reacts according to reaction (3) in the CPO reactor 100 is less than the amount of methane that reacts according to reaction (1) in the CPO reactor 100. In an aspect, less than about 50 mol %, alternatively less than about 40 mol %, alternatively less than about 30 mol %, alternatively less than about 20 mol %, or alternatively less than about 10 mol % of hydrocarbons (e.g., methane) react with steam in the CPO reactor 100.

Without wishing to be limited by theory, the presence of water and/or steam in the CPO reactor 100 changes the flammability of the CPO reactant mixture 10, thereby providing for a wider practical range of C/O molar ratios in the CPO reactant mixture 10. Further, and without wishing to be limited by theory, the presence of water and/or steam in the CPO reactor 100 allows for the use of lower C/O molar ratios in the CPO reactant mixture 10. Furthermore, and without wishing to be limited by theory, the presence of water and/or steam in the CPO reactor 100 allows for operating the CPO reactor 100 at relatively high pressures.

In an aspect, the CPO reactor 100 can be operated in the presence of water and/or steam at a CPO pressure of equal to or greater than about 10 barg, alternatively equal to or greater than about 20 barg, alternatively equal to or greater than about 25 barg, alternatively equal to or greater than about 30 barg, alternatively equal to or greater than about 35 barg, alternatively equal to or greater than about 40 barg, alternatively equal to or greater than about 50 barg.

In an aspect, the CPO reactor 100 can be operated in the presence of water and/or steam at a C/O molar ratio in the CPO reactant mixture 10 of less than about 2.2:1, alternatively less than about 2:1, alternatively less than about 1.9:1, alternatively from about 0.9:1 to about 2.2:1, alternatively from about 1:1 to about 2:1, or alternatively from about 1.1:1 to about 1.9:1.

As will be appreciated by one of skill in the art, and with the help of this disclosure, the introduction of water and/or steam in the CPO reactor 100 can lead to increasing the amount of unreacted hydrocarbons in the CPO reactor effluent 15 and/or syngas 20. Further, as will be appreciated by one of skill in the art, and with the help of this disclosure, methanol production processes typically tolerate limited amounts of unreacted hydrocarbons in the syngas.

In some aspects, the CPO reactor effluent 15 and/or syngas 20 can comprise less than about 7.5 mol %, alternatively less than about 5 mol %, or alternatively less than about 2.5 mol % hydrocarbons (e.g., unreacted hydrocarbons, unreacted CH₄). In such aspects, the CPO reactor effluent 15 and/or syngas 20 can be produced in a CPO process that employs water and/or steam. In such aspects, the CPO reactor effluent 15 and/or syngas 20 can be used for methanol synthesis.

In some aspects, the CPO reactor 100 can be operated at an S/C molar ratio in the CPO reactant mixture of less than about 1:1, at a CPO pressure of less than about 30 barg, and at a C/O molar ratio in the CPO reactant mixture 10 of less than about 2.2:1.

In an aspect, a process for producing methanol as disclosed herein can comprise (i) recovering a CPO reactor effluent 15 from the CPO reactor 100, wherein the CPO reactor effluent 15 comprises hydrogen, carbon monoxide, carbon dioxide, water, and unreacted hydrocarbons; and (ii) processing at least a portion of the CPO reactor effluent 15 to produce the syngas 20; wherein (1) the CO₂ content of the syngas 20 is lower than the CO₂ content of the CPO reactor effluent 15; and/or (2) the H₂ content of the syngas 20 is greater than the H₂ content of the CPO reactor effluent 15. As will be appreciated by one of skill in the art, and with the help of this disclosure, even if the reactor effluent (e.g., CPO reactor effluent 15) recovered from the CPO reactor 100 is characterized by (1) a CO₂ content of from about 0.1 mol % to about 5 mol %, and/or (2) a H₂/CO molar ratio of greater than about 1.9, the reactor effluent can be further processed to decrease the CO₂ content and/or enrich the hydrogen content of the reactor effluent to provide for a syngas with a desired composition.

In an aspect, the step of processing at least a portion of the CPO reactor effluent 15 to produce the syngas 20 can comprise removing at least a portion of the carbon dioxide from the CPO reactor effluent 15 to yield the syngas 20. As will be appreciated by one of skill in the art, and with the help of this disclosure, and without wishing to be limited by theory, while the H₂/CO molar ratio of the syngas does not change by removing carbon dioxide from the syngas, the concentration of hydrogen increases in the syngas by removing carbon dioxide from the syngas. However, the M ratio of the syngas changes with changing the carbon dioxide content of the syngas, wherein the M ratio is a molar ratio defined as (H₂—CO₂)/(CO+CO₂). The CPO reactor effluent 15 is characterized by an M ratio of the CPO reactor effluent 15. The syngas 20 is characterized by an M ratio of the syngas 20. In aspects where the syngas 20 is produced by removing at least a portion of the carbon dioxide from the CPO reactor effluent 15, the syngas 20 can be characterized by an M ratio that is greater than the M ratio of the CPO reactor effluent 15.

In an aspect, the CPO reactor effluent 15 can be characterized by an M ratio of from about 1.2 to about 1.8, alternatively from about 1.6 to about 1.78, or alternatively from about 1.7 to about 1.78.

In some aspects, at least a portion of the CPO reactor effluent 15 can be introduced to the CO₂ separator 150 (e.g., CO₂ scrubber) to yield the syngas 20, wherein the syngas 20 can be characterized by an M ratio that is greater than the M ratio of the CPO reactor effluent 15. The CO₂ separator 150 can comprise CO₂ removal by amine (e.g., monoethanolamine) absorption (e.g., amine scrubbing), pressure swing adsorption (PSA), temperature swing adsorption, gas separation membranes (e.g., porous inorganic membranes, palladium membranes, polymeric membranes, zeolites, etc.), cryogenic separation, and the like, or combinations thereof. In an aspect, the step of removing at least a portion of the carbon dioxide from the CPO reactor effluent 15 to yield the syngas 20 can comprise CO₂ removal by amine absorption. As will be appreciated by one of skill in the art, and with the help of this disclosure, a CO₂-lean syngas has a higher M ratio than a CO₂-rich syngas: the lower the CO₂ content of the syngas, the higher the M ratio of the syngas.

In an aspect, the syngas 20 can be characterized by an M ratio of from about 1.9 to about 2.2, alternatively from about 1.95 to about 2.1, or alternatively from about 1.98 to about 2.06.

As will be appreciated by one of skill in the art, and with the help of this disclosure, if the CPO reactor effluent 15 has a CO₂ content of from about 0.1 mol % to about 5 mol %, the step of removing at least a portion of the carbon dioxide from the CPO reactor effluent 15 to yield the syngas 20 can be performed, but is not necessary. For example, side reactions as represented by equations (7) and/or (8) could lead to a CPO reactor effluent 15 that has a CO₂ content of from about 0.1 mol % to about 5 mol %.

In an aspect, the CPO reactor effluent 15 and/or syngas 20 can have a CO₂ content of of less than about 5 mol %, alternatively less than about 4 mol %, alternatively less than about 3 mol %, alternatively less than about 2 mol %, alternatively less than about 1 mol %, alternatively from about 0.1 mol % to about 5 mol %, alternatively from about 0.25 mol % to about 4 mol %, or alternatively from about 0.5 mol % to about 3 mol %.

In an aspect, the CPO reactor effluent 15 and/or syngas 20 can be characterized by a carbon monoxide to carbon dioxide (CO/CO₂) molar ratio of equal to or greater than about 5, alternatively equal to or greater than about 7.5, alternatively equal to or greater than about 10, alternatively equal to or greater than about 12.5, or alternatively equal to or greater than about 15.

The CO₂ content of the syngas (e.g., CPO reactor effluent 15 and/or syngas 20) can be adjusted as described in more detail in the co-pending U.S. Provisional Patent Application No. 62/787,574 and entitled “Hydrogen Enrichment in Syngas Produced via Catalytic Partial Oxidation”); which is incorporated by reference herein in its entirety.

In an aspect, the step of processing at least a portion of the CPO reactor effluent 15 to produce the syngas 20 can comprise contacting an SMR reactor syngas effluent 12 with at least a portion of the CPO reactor effluent 15 and/or at least a portion of the syngas 20 prior to introducing the CPO reactor effluent 15 and/or the syngas 20 to the methanol reactor 200, respectively; wherein the SMR reactor syngas effluent 12 can increase the H₂ content of the CPO reactor effluent 15 and/or the syngas 20, respectively.

In an aspect, at least a portion 12a of the SMR reactor syngas effluent 12 can be contacted with at least a portion of the CPO reactor effluent 15 to yield the syngas 20.

In an aspect, at least a portion 12c of the SMR reactor syngas effluent 12 can be contacted with at least a portion of a CO₂ separator effluent to yield the syngas 20.

The SMR reactor syngas effluent 12 can be produced by reacting, via an SMR reaction (e.g., a reaction represented by equation (3)), an SMR reactant mixture 11 in the SMR reactor 110 to produce the SMR reactor syngas effluent 12; wherein the SMR reactant mixture 11 comprises methane and steam; and wherein the SMR reactor syngas effluent 12 comprises hydrogen, carbon monoxide, carbon dioxide, water, and unreacted methane. Generally, SMR describes the catalytic reaction of methane and steam to form carbon monoxide and hydrogen according to the reaction represented by equation (3). Steam reforming catalysts can comprise any suitable commercially available steam reforming catalyst; nickel (Ni) and/or rhodium (Rh) as active metal(s) on alumina; or combinations thereof. SMR employs fairly elevated S/C molar ratios when compared to the S/C molar ratios used in CPO. For example, SMR can be characterized by an S/C molar ratio of equal to or greater than about 2.5, alternatively equal to or greater than about 2.7, or alternatively equal to or greater than about 3.0. Further, the SMR reactor syngas effluent 12 can be characterized by a H₂/CO molar ratio of equal to or greater than about 2.5, alternatively equal to or greater than about 2.7, or alternatively equal to or greater than about 2.9. As will be appreciated by one of skill in the art, and with the help of this disclosure, and without wishing to be limited by theory, the SMR reaction as represented by equation (3) can yield a syngas with a H₂/CO molar ratio having the theoretical stoichiometric limit of 3.0 (i.e., SMR reaction as represented by equation (3) yields 3 moles of H₂ for every 1 mole of CO). As will be appreciated by one of skill in the art, and with the help of this disclosure, the theoretical stoichiometric limit of 3.0 for the H₂/CO molar ratio in an SMR reaction cannot be achieved because reactants undergo side reactions at the conditions used for the SMR reaction. The M ratio of the SMR reactor syngas effluent 12 is greater than the M ratio of the CPO reactor effluent 15.

In some aspects, at least a portion 12b of the SMR reactor syngas effluent 12 can be fed to the CPO reactor 100 to produce the CPO reactor effluent 15. In such aspects, the SMR reactor syngas effluent 12 comprises unreacted hydrocarbons (e.g., CH₄) that can participate in the CPO reaction as represented by equation (1). Since the SMR reactor syngas effluent 12 has a fairly high H₂/CO molar ratio (e.g., equal to or greater than about 2.5), the syngas recovered from the CPO reactor can have a H₂/CO molar ratio that is greater than the H₂/CO molar ratio of a syngas produced via an otherwise similar CPO process without feeding an SMR reactor syngas effluent 12 to the CPO reactor 100.

In aspects where the CPO reactor effluent 15 and/or the syngas 20 is characterized by an M ratio of from about 1.8 to about 2.2, the CPO reactor effluent 15 and/or the syngas 20 can be further used for methanol production.

In an aspect, a process for producing methanol as disclosed herein can comprise a step of introducing at least a portion of the CPO reactor effluent 15 and/or the syngas 20 to the methanol reactor 200 to produce a methanol reactor effluent stream 30; wherein the methanol reactor effluent stream 30 comprises methanol, water, hydrogen, carbon monoxide, carbon dioxide, and hydrocarbons. The methanol reactor 200 can comprise any reactor suitable for a methanol synthesis reaction from CO and H₂, such as for example an isothermal reactor, an adiabatic reactor, a trickle bed reactor, a fluidized bed reactor, a slurry reactor, a loop reactor, a cooled multi tubular reactor, and the like, or combinations thereof.

Generally, CO and H₂ can be converted into methanol (CH₃OH), for example as represented by equation (9):

CO+H₂ [symbol] CH₃OH (9)

CO₂ and H₂ can also be converted to methanol, for example as represented by equation (10):

CO₂+3H₂ [symbol] CH₃OH+H₂O (10)

Without wishing to be limited by theory, the lower the CO₂ content of the CPO reactor effluent 15 and/or the syngas 20, the lower the amount of water produced in the methanol reactor 200. As will be appreciated by one of skill in the art, and with the help of this disclosure, syngas produced by SMR has a fairly high content of hydrogen (as compared to the hydrogen content of syngas produced by CPO), and a syngas with an elevated hydrogen content can promote the CO₂ conversion to methanol, for example as represented by equation (10), which in turn can lead to an increased water content in a crude methanol stream (e.g., crude methanol stream 40).

Methanol synthesis from CO, CO₂ and H₂ is a catalytic process, and is most often conducted in the presence of copper based catalysts. The methanol reactor 200 can comprise a methanol production catalyst, such as any suitable commercial catalyst used for methanol synthesis. Nonlimiting examples of methanol production catalysts suitable for use in the methanol reactor 200 in the current disclosure include Cu, Cu/ZnO, Cu/ThO₂, Cu/Zn/Al₂O₃, Cu/ZnO/Al₂O₃, Cu/Zr, and the like, or combinations thereof.

In an aspect, a process for producing methanol as disclosed herein can comprise a step of separating at least a portion of the methanol reactor effluent stream 30 into a crude methanol stream 40 and a vapor stream 50; wherein the crude methanol stream 40 comprises methanol and water; wherein the vapor stream 50 comprises hydrogen, carbon monoxide, carbon dioxide, and hydrocarbons. The methanol reactor effluent stream 30 can be separated into the crude methanol stream 40 and the vapor stream 50 in the gas-liquid separator 300, such as a vapor-liquid separator, flash drum, knock-out drum, knock-out pot, compressor suction drum, etc.

In an aspect, the crude methanol stream 40 can comprise water in an amount of less than about 10 wt. %, alternatively less than about 8 wt. %, alternatively less than about 6 wt. %, alternatively less than about 4 wt. %, alternatively less than about 3 wt. %, alternatively less than about 2 wt. %, or alternatively less than about 1 wt. %, based on the total weight of the crude methanol stream 40.

In an aspect, the crude methanol stream 40 can comprise methanol in an amount of equal to or greater than about 90 wt. %, alternatively equal to or greater than about 92 wt. %, alternatively equal to or greater than about 94 wt. %, alternatively equal to or greater than about 96 wt. %, alternatively equal to or greater than about 97 wt. %, alternatively equal to or greater than about 98 wt. %, or alternatively equal to or greater than about 99 wt. %, based on the total weight of the crude methanol stream 40.

In an aspect, a process for producing methanol as disclosed herein can comprise a step of separating at least a portion of the crude methanol stream 40 in the distillation unit 400 into a methanol stream 45 and a water stream 46, wherein the distillation unit 400 comprises one or more distillation columns. The water stream 46 comprises water and residual methanol. Generally, the one or more distillation columns can separate components of the crude methanol stream 40 based on their boiling points. As will be appreciated by one of skill in the art, and with the help of this disclosure, the higher the water content of the crude methanol stream 40, the more energy will be expanded in the distillation unit to purify the methanol.

In an aspect, the methanol stream 45 can comprise methanol in an amount of equal to or greater than about 95 wt. %, alternatively equal to or greater than about 97.5 wt. %, alternatively equal to or greater than about 99 wt. %, or alternatively equal to or greater than about 99.9 wt. %, based on the total weight of the methanol stream 45.

In an aspect, a process for producing methanol as disclosed herein can comprise a step of separating at least a portion of the vapor stream 50 into a hydrogen stream 51 and a residual gas stream 52, wherein the hydrogen stream 51 comprises at least a portion of the hydrogen of the vapor stream 50, and wherein the residual gas stream 52 comprises carbon monoxide, carbon dioxide, and hydrocarbons. The vapor stream 50 can be separated into the hydrogen stream 51 and the residual gas stream 52 in a hydrogen recovery unit 500, such as a PSA unit, a membrane separation unit, a cryogenic separation unit, and the like, or combinations thereof.

In an aspect, at least a portion of the residual gas stream 52 can be purged. In an aspect, at least a portion of the residual gas stream 52 can be used as fuel, for example for pre-heating the CPO reactant mixture 10 and/or the SMR reactor 110.

In an aspect, a process for producing methanol as disclosed herein can comprise recycling at least a portion 51a of the hydrogen stream 51 to the methanol reactor 200; for example via CPO reactor effluent 15 and/or syngas 20.

In an aspect, a process for producing methanol can comprise the steps of (a) reacting, via a catalytic partial oxidation (CPO) reaction, a CPO reactant mixture 10 in a CPO reactor 100 to produce a CPO reactor effluent 15; wherein the CPO reactant mixture 10 comprises hydrocarbons, oxygen, and optionally steam; wherein the CPO reactor 100 comprises a CPO catalyst; wherein the CPO reactor effluent 15 comprises hydrogen, carbon monoxide, carbon dioxide, water, and unreacted hydrocarbons; (b) cooling at least a portion of the CPO reactor effluent 15 to yield a cooled CPO reactor effluent and process heat (e.g., which can be recovered and used as thermal energy); (c) removing at least a portion of the water from the cooled CPO reactor effluent to yield a dehydrated CPO reactor effluent, wherein the dehydrated CPO reactor effluent comprises H₂, CO, CO₂, and unreacted hydrocarbons; (d) removing at least a portion of the carbon dioxide from the dehydrated CPO reactor effluent in a CO₂ separator 150 to yield syngas 20, wherein the syngas 20 comprises carbon dioxide in an amount of from about 0.1 mol % to about 5 mol %; (e) introducing at least a portion of the syngas 20 to a methanol reactor 200 to produce a methanol reactor effluent stream 30; wherein the methanol reactor effluent stream 30 comprises methanol, water, hydrogen, carbon monoxide, carbon dioxide, and hydrocarbons; (f) separating at least a portion of the methanol reactor effluent stream 30 in a gas-liquid separator 300 into a crude methanol stream 40 and a vapor stream 50, wherein the crude methanol stream 40 comprises methanol and water, wherein the vapor stream 50 comprises hydrogen, carbon monoxide, carbon dioxide, and hydrocarbons; and wherein the crude methanol stream 40 comprises water in an amount of less than about 5 wt. %, based on the total weight of the crude methanol stream 40; (g) separating at least a portion of the crude methanol stream 40 in a distillation unit 400 into a methanol stream 45 and a water stream 46, wherein the distillation unit comprises one or more distillation columns; (h) separating at least a portion of the vapor stream 50 in a hydrogen recovery unit 500 into a hydrogen stream 51 and a residual gas stream 52, wherein the hydrogen stream 51 comprises at least a portion of the hydrogen of the vapor stream 50, and wherein the residual gas stream 52 comprises carbon monoxide, carbon dioxide, and hydrocarbons; and (i) recycling at least a portion 51a of the hydrogen stream 51 to the methanol reactor 200. In such aspect, the CPO reactor 100 is characterized by a S/C molar ratio in the CPO reactant mixture 10 of less than about 0.5:1; wherein a portion of the hydrocarbons in the CPO reactant mixture 10 undergo decomposition to carbon and hydrogen, wherein at least a portion of the carbon reacts with carbon dioxide in the CPO reactor 100 to produce carbon monoxide, and/or wherein at least a portion of the carbon reacts with water in the CPO reactor 100 to produce carbon monoxide and hydrogen.

In an aspect, a process for producing methanol as disclosed herein can advantageously display improvements in one or more process characteristics when compared to an otherwise similar process that introduces to a methanol reactor a syngas comprising carbon dioxide in an amount of equal to or greater than about 5 mol %. The process for producing methanol as disclosed herein can advantageously reduce the overall energy consumption in methanol production by reducing the water content in the crude methanol. The process for producing methanol as disclosed herein can advantageously reduce the water content in the crude methanol by reducing the CO₂ content of the syngas that is introduced to the methanol reactor.

As will be appreciated by one of skill in the art, and with the help of this disclosure, the quality of syngas (e.g., the syngas composition) that is fed to a specific process (e.g., methanol production process) can have an important impact on the stream flow rates, as well as product selectivity. For example, in the case of a methanol production process, the syngas composition used for producing methanol can change a composition of the crude methanol recovered from a methanol production reactor (e.g., a loop reactor), wherein the crude methanol can be rich in methanol (as opposed to rich in water); thereby advantageously changing the process downstream of the methanol reactor, owing to reduced recycle streams (due to having only the necessary amount of hydrogen in the syngas), as well as to a reduced amount water in the crude methanol product. Thus, the methanol production process can advantageously be more energy efficient; owing to a lower energy consumption in a methanol purification section. Since the CO₂ amount in the syngas is reduced (for example by comparison with a syngas comprising carbon dioxide in an amount of equal to or greater than about 5 mol %), recycle flow loops would be of smaller size and recycle gas compressors needed would be of smaller volumetric flow rate and thus consume less electricity. The methanol production process can advantageously be more carbon efficient, by saving hydrocarbon feedstock (e.g., natural gas) employed in the production of the syngas (e.g., less carbon gets converted to CO₂). For purposes of the disclosure herein the carbon efficiency is defined as the ratio of the number of moles of carbon present in the methanol stream (e.g., methanol stream 45) to the number of moles of carbon in the CPO reactant mixture (e.g., CPO reactant mixture 10).

In an aspect, a process for producing methanol as disclosed herein can advantageously provide for an on-stream factor of the methanol reactor that is greater than the on-stream factor of a methanol reactor in an otherwise similar process that introduces to the methanol reactor a syngas comprising carbon dioxide in an amount of equal to or greater than about 5 mol %. For purposes of the disclosure herein the on-stream factor is defined as the ratio of the number of days in a year that a reactor is actively producing a desired product to the number of days in a calendar year.

In an aspect, a process for producing methanol as disclosed herein can advantageously allow for controlling the composition of the syngas produced via CPO (e.g., by controlling CPO operational parameters), which in turn can advantageously lead to a decreased water content of the crude methanol stream.

In some aspects, SMR can be advantageously used in conjunction with CPO as disclosed herein to provide for a syngas having a composition that can advantageously lead to a decreased water content of the crude methanol stream.

As will be appreciated by one of skill in the art, and with the help of this disclosure, since the CPO reaction is exothermic, no additional heat supply in the form of fuel combustion is needed (except for pre-heating reactants in the reaction mixture that is supplied to a syngas generation section), when compared to conventional steam reforming. As such, the process for producing syngas as disclosed herein can advantageously generate less CO₂ through fuel burning, when compared to steam reforming. Additional advantages of the processes for the production of methanol as disclosed herein can be apparent to one of skill in the art viewing this disclosure.

### EXAMPLES

The subject matter having been generally described, the following examples are given as particular embodiments of the disclosure and to demonstrate the practice and advantages thereof. It is understood that the examples are given by way of illustration and are not intended to limit the specification of the claims to follow in any manner.

### Example 1

The water content in a methanol production system was investigated based on the composition of the syngas used for methanol synthesis. A conventional method of producing syngas via combined reforming (CR) technology that pairs steam methane reforming (SMR) with autothermal reforming (ATR) was compared to the method of producing syngas via CPO, wherein each type of syngas (i.e., from CR and CPO) was further converted to methanol.

For the CR technology, the syngas was produced by a conventional process.

For CPO, the syngas was produced with two different preheating temperatures for the reactant mixture. Process conditions were varied as would be understood by one of skill in the art. For example, reaction temperatures were from about 800° C. to about 1,100° C.

Methanol was produced by a conventional technology, and the water content of the crude methanol stream is displayed in Table 1 for all 3 cases.

TABLE 1

| | | CPOx | CPOx |
|---|---|---|---|
| | | Reactant mixture | Reactant mixture |
| | SMR + | preheat temperature | preheat temperature |
| | ATR | of 350° C. | of 520° C. |
| Overall Carbon | 77% | 88% | 91% |
| Efficiency | | | |
| S/C ratio for syngas | 1.5 | 0.2 | 0.2 |
| production | | | |
| Gas Flow (MMscmh) | 0.6 | 0.46 | 0.47 |
| Overall CO₂ emission | 23% | 12% | 9% |
| Water in | 20% | 4% | 3% |
| MeOH [wt. %] | | | |
| CO₂ in Syngas | 7% | 3% | 2.5% |
| [mol %] | | | |
| CO/CO₂ molar ratio | 3 | 10 | 12 |
| of Syngas | | | |
| M Value of Syngas | 2.5 | 1.82 | 1.83 |

The data in Table 1 indicate that by using CPO for the production of syngas, the water content in the crude methanol stream can be reduced from 20 wt. % (CR) to 3-4 wt. % (CPO). The water content reduction correlates with the reduction in CO₂ content of syngas from 7 mol % (CR) to 2.5-3 mol % (CPO), respectively. Further, the overall carbon efficiency goes up with decreasing the CO₂ content of syngas.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. § 1.72 and the purpose stated in 37 C.F.R. § 1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

### ADDITIONAL DISCLOSURE

The following are non-limiting, specific embodiments in accordance with the present disclosure:

A first embodiment, which is a process for producing methanol comprising (a) reacting, via a catalytic partial oxidation (CPO) reaction, a CPO reactant mixture in a CPO reactor to produce syngas; wherein the CPO reactant mixture comprises hydrocarbons and oxygen; wherein the CPO reactor comprises a CPO catalyst; and wherein the syngas comprises hydrogen, carbon monoxide, carbon dioxide, water, and unreacted hydrocarbons, (b) introducing at least a portion of the syngas to a methanol reactor to produce a methanol reactor effluent stream; wherein the methanol reactor effluent stream comprises methanol, water, hydrogen, carbon monoxide, carbon dioxide, and hydrocarbons, and (c) separating at least a portion of the methanol reactor effluent stream into a crude methanol stream and a vapor stream; wherein the crude methanol stream comprises methanol and water; wherein the vapor stream comprises hydrogen, carbon monoxide, carbon dioxide, and hydrocarbons; and wherein the crude methanol stream comprises water in an amount of less than about 10 wt. %, based on the total weight of the crude methanol stream.

A second embodiment, which is the process of the first embodiment, wherein the syngas comprises carbon dioxide in an amount of from about 0.1 mol % to about 5 mol %.

A third embodiment, which is the process of any of the first through the second embodiments, wherein the syngas is characterized by a carbon monoxide to carbon dioxide (CO/CO₂) molar ratio of equal to or greater than about 5.

A fourth embodiment, which is the process of any of the first through the third embodiments, wherein the hydrocarbons comprise methane, natural gas, natural gas liquids, associated gas, well head gas, enriched gas, paraffins, shale gas, shale liquids, fluid catalytic cracking (FCC) off gas, refinery process gases, stack gases, fuel gas from fuel gas header, or combinations thereof.

A fifth embodiment, which is the process of any of the first through the fourth embodiments, wherein the CPO reactor is characterized by a steam to carbon (S/C) molar ratio in the CPO reactant mixture of from about 0.01:1 to less than about 2.4:1.

A sixth embodiment, which is the process of any of the first through the fifth embodiments further comprising (i) recovering a CPO reactor effluent from the CPO reactor, wherein the CPO reactor effluent comprises hydrogen, carbon monoxide, carbon dioxide, water, and unreacted hydrocarbons, and wherein the amount of carbon dioxide in the CPO reactor effluent is greater than the amount of carbon dioxide in the syngas; and (ii) removing at least a portion of the carbon dioxide from the CPO reactor effluent to yield the syngas.

A seventh embodiment, which is the process of the sixth embodiment, wherein the CPO reactor effluent is characterized by a M ratio of the CPO reactor effluent, wherein the M ratio is a molar ratio defined as (H₂—CO₂)/(CO+CO₂); and wherein the syngas is characterized by an M ratio that is greater than the M ratio of the CPO reactor effluent.

An eighth embodiment, which is the process of the seventh embodiment further comprising reacting, via a steam methane reforming (SMR) reaction, an SMR reactant mixture in an SMR reactor to produce an SMR reactor syngas effluent; wherein the SMR reactant mixture comprises methane and steam; wherein the SMR reactor syngas effluent comprises hydrogen, carbon monoxide, carbon dioxide, water, and unreacted methane; and wherein the M ratio of the SMR reactor syngas effluent is greater than the M ratio of the CPO reactor effluent.

A ninth embodiment, which is the process of the eighth embodiment further comprising contacting at least a portion of the SMR reactor syngas effluent with at least a portion of the CPO reactor effluent to yield the syngas.

A tenth embodiment, which is the process of the eighth embodiment further comprising introducing at least a portion of the SMR reactor syngas effluent to the CPO reactor.

An eleventh embodiment, which is the process of the eighth embodiment, wherein the S/C molar ratio in the SMR reactant mixture is greater than the S/C molar ratio in the CPO reactant mixture, wherein the S/C molar ratio refers to the total moles of water (H₂O) in the reactant mixture divided by the total moles of carbon (C) of hydrocarbons in the reactant mixture.

A twelfth embodiment, which is the process of any of the first through the eleventh embodiments, wherein the CPO reactor is characterized by at least one CPO operational parameter selected from the group consisting of a CPO feed temperature of from about 25° C. to about 600° C.; a CPO effluent temperature of from about 300° C. to about 1,600° C.; a CPO pressure of from about 1 barg to about 90 barg; a CPO contact time of from about 0.001 milliseconds (ms) to about 5 s; a carbon to oxygen (C/O) molar ratio in the CPO reactant mixture of from about 0.5:1 to about 3:1, wherein the C/O molar ratio refers to the total moles of carbon (C) of hydrocarbons in the reactant mixture divided by the total moles of oxygen (O₂) in the reactant mixture; and combinations thereof.

A thirteenth embodiment, which is the process of the twelfth embodiment, wherein the at least one operational parameter comprises a steam to carbon (S/C) molar ratio in the CPO reactant mixture of less than about 1:1, wherein the S/C molar ratio refers to the total moles of water (H₂O) in the reactant mixture divided by the total moles of carbon (C) of hydrocarbons in the reactant mixture.

A fourteenth embodiment, which is the process of any of the twelfth through the thirteenth embodiments, wherein the at least one operational parameter comprises a CPO pressure of less than about 30 barg.

A fifteenth embodiment, which is the process of any of the twelfth through the fourteenth embodiments, wherein the at least one operational parameter comprises a CPO effluent temperature of equal to or greater than about 750° C. and/or a C/O molar ratio in the CPO reactant mixture of less than about 2.2:1.

A sixteenth embodiment, which is the process of any of the first through the fifteenth embodiments, wherein a portion of the hydrocarbons in the CPO reactant mixture undergo decomposition to carbon and hydrogen, and wherein at least a portion of the carbon reacts with carbon dioxide in the CPO reactor to produce carbon monoxide.

A seventeenth embodiment, which is the process of any of the first through the sixteenth embodiments further comprising (i) separating at least a portion of the vapor stream into a hydrogen stream and a residual gas stream, wherein the hydrogen stream comprises at least a portion of the hydrogen of the vapor stream, and wherein the residual gas stream comprises carbon monoxide, carbon dioxide, and hydrocarbons; and (ii) recycling at least a portion of the hydrogen stream to the methanol reactor.

An eighteenth embodiment, which is a process for producing methanol comprising (a) reacting, via a catalytic partial oxidation (CPO) reaction, a CPO reactant mixture in a CPO reactor to produce a CPO reactor effluent; wherein the CPO reactant mixture comprises hydrocarbons and oxygen; wherein the CPO reactor comprises a CPO catalyst; wherein the CPO reactor effluent comprises hydrogen, carbon monoxide, carbon dioxide, water, and unreacted hydrocarbons, (b) removing at least a portion of the carbon dioxide from the CPO reactor effluent in a carbon dioxide separator to yield syngas, wherein the syngas comprises carbon dioxide in an amount from about 0.1 mol % to about 5 mol %, (c) introducing at least a portion of the syngas to a methanol reactor to produce a methanol reactor effluent stream; wherein the methanol reactor effluent stream comprises methanol, water, hydrogen, carbon monoxide, carbon dioxide, and hydrocarbons, (d) separating at least a portion of the methanol reactor effluent stream into a crude methanol stream and a vapor stream, wherein the crude methanol stream comprises methanol and water, wherein the vapor stream comprises hydrogen, carbon monoxide, carbon dioxide, and hydrocarbons; and wherein the crude methanol stream comprises water in an amount of less than about 5 wt. %, based on the total weight of the crude methanol stream, (e) separating at least a portion of the crude methanol stream in a distillation unit into a methanol stream and a water stream, wherein the distillation unit comprises one or more distillation columns, (f) separating at least a portion of the vapor stream into a hydrogen stream and a residual gas stream, wherein the hydrogen stream comprises at least a portion of the hydrogen of the vapor stream, and wherein the residual gas stream comprises carbon monoxide, carbon dioxide, and hydrocarbons, and (g) recycling at least a portion of the hydrogen stream to the methanol reactor.

A nineteenth embodiment, which is the process of the eighteenth embodiment, wherein the CPO reactor is characterized by a steam to carbon (S/C) molar ratio in the CPO reactant mixture of less than about 0.5:1, wherein the S/C molar ratio refers to the total moles of water (H₂O) in the reactant mixture divided by the total moles of carbon (C) of hydrocarbons in the reactant mixture; wherein a portion of the hydrocarbons in the CPO reactant mixture undergo decomposition to carbon and hydrogen, wherein at least a portion of the carbon reacts with carbon dioxide in the CPO reactor to produce carbon monoxide and/or wherein at least a portion of the carbon reacts with water in the CPO reactor to produce carbon monoxide and hydrogen.

A twentieth embodiment, which is the process of any of the eighteenth through the nineteenth embodiments further comprising (1) cooling at least a portion of the CPO reactor effluent to yield a cooled CPO reactor effluent; (2) removing at least a portion of the water from the cooled CPO reactor effluent to yield a dehydrated CPO reactor effluent, wherein the dehydrated CPO reactor effluent comprises hydrogen, carbon monoxide, carbon dioxide, and unreacted hydrocarbons; and (3) feeding at least a portion of the dehydrated CPO reactor effluent to the carbon dioxide separator in step (b).

While embodiments of the disclosure have been shown and described, modifications thereof can be made without departing from the spirit and teachings of the invention. The embodiments and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

1. A process for producing methanol comprising:
(a) reacting, via a catalytic partial oxidation (CPO) reaction, a CPO reactant mixture in a CPO reactor to produce syngas; wherein the CPO reactant mixture comprises hydrocarbons and oxygen; wherein the CPO reactor comprises a CPO catalyst;
and wherein the syngas comprises hydrogen, carbon monoxide, carbon dioxide, water, and unreacted hydrocarbons;
(b) introducing at least a portion of the syngas to a methanol reactor to produce a methanol reactor effluent stream; wherein the methanol reactor effluent stream comprises methanol, water, hydrogen, carbon monoxide, carbon dioxide, and hydrocarbons; and
(c) separating at least a portion of the methanol reactor effluent stream into a crude methanol stream and a vapor stream; wherein the crude methanol stream comprises methanol and water; wherein the vapor stream comprises hydrogen, carbon monoxide, carbon dioxide, and hydrocarbons; and wherein the crude methanol stream comprises water in an amount of less than about 10 wt. %, based on the total weight of the crude methanol stream.

2. The process of claim 1, wherein the syngas comprises carbon dioxide in an amount of from about 0.1 mol % to about 5 mol %.

3. The process of claim 1, wherein the syngas includes a carbon monoxide to carbon dioxide (C0/C02) molar ratio of equal to or greater than about 5.

4. The process of claim 1, wherein the hydrocarbons comprise methane, natural gas, natural gas liquids, associated gas, well head gas, enriched gas, paraffins, shale gas, shale liquids, fluid catalytic cracking (FCC) off gas, refinery process gases, stack gases, fuel gas from fuel gas header, or combinations thereof.

5. The process of claim 1, wherein the CPO reactor includes a steam to carbon (S/C) molar ratio in the CPO reactant mixture of from about 0.01 :1 to less than about 2.4:1.

6. The process of claim 1 further comprising the steps of: (i) recovering a CPO reactor effluent from the CPO reactor, wherein the CPO reactor effluent comprises hydrogen, carbon monoxide, carbon dioxide, water, and unreacted hydrocarbons, and wherein the amount of carbon dioxide in the CPO reactor effluent is greater than the amount of carbon dioxide in the syngas; and (ii) removing at least a portion of the carbon dioxide from the CPO reactor effluent to yield the syngas.

7. The process of claim 6, wherein the CPO reactor effluent includes a M ratio of the CPO reactor effluent, wherein the M ratio is a molar ratio defined as (H2−C02)/(C0+C02); and wherein the syngas includes an M ratio that is greater than the M ratio of the CPO reactor effluent.

8. The process of claim 7 further comprising the step of: reacting, via a steam methane reforming (SMR) reaction, an SMR reactant mixture in an SMR reactor to produce an SMR reactor syngas effluent; wherein the SMR reactant mixture comprises methane and steam; wherein the SMR reactor syngas effluent comprises hydrogen, carbon monoxide, carbon dioxide, water, and unreacted methane;
and wherein the M ratio of the SMR reactor syngas effluent is greater than the M ratio of the CPO reactor effluent.

9. The process of claim 8 further comprising the step of contacting at least a portion of the SMR reactor syngas effluent with at least a portion of the CPO reactor effluent to yield the syngas.

10. The process of claim 8 further comprising the step of introducing at least a portion of the SMR reactor syngas effluent to the CPO reactor.

11. The process of claim 8, wherein the S/C molar ratio in the SMR reactant mixture is greater than the S/C molar ratio in the CPO reactant mixture, wherein the S/C molar ratio refers to the total moles of water (H20) in the reactant mixture divided by the total moles of carbon (C) of hydrocarbons in the reactant mixture.

12. The process of claim 1, wherein the CPO reactor includes at least one CPO operational parameter selected from the group consisting of a CPO feed temperature of from about 25° C. to about 600° C.; a CPO effluent temperature of from about 300° C. to about 1,600° C.; a CPO pressure of from about 1 barg to about 90 barg; a CPO contact time of from about 0.001 milliseconds (ms) to about 5 s; a carbon to oxygen (C/O) molar ratio in the CPO reactant mixture of from about 0.5:1 to about 3:1, wherein the C/O molar ratio refers to the total moles of carbon (C) of hydrocarbons in the reactant mixture divided by the total moles of oxygen (02) in the reactant mixture; and combinations thereof.

13. The process of claim 12, wherein the at least one operational parameter comprises a steam to carbon (S/C) molar ratio in the CPO reactant mixture of less than about 1:1, wherein the S/C molar ratio refers to the total moles of water (H20) in the reactant mixture divided by the total moles of carbon (C) of hydrocarbons in the reactant mixture.

14. The process of claim 12, wherein the at least one operational parameter comprises a CPO pressure of less than about 30 barg.

15. The process of claim 12, wherein the at least one operational parameter comprises a CPO effluent temperature of equal to or greater than about 750° C. and/or a C/O molar ratio in the CPO reactant mixture of less than about 2.2:1.

16. The process of claim 1, wherein a portion of the hydrocarbons in the CPO reactant mixture undergo decomposition to carbon and hydrogen, and wherein at least a portion of the carbon reacts with carbon dioxide in the CPO reactor to produce carbon monoxide.

17. The process of claim 1 further comprising the steps of: (i) separating at least a portion of the vapor stream into a hydrogen stream and a residual gas stream, wherein the hydrogen stream comprises at least a portion of the hydrogen of the vapor stream, and wherein the residual gas stream comprises carbon monoxide, carbon dioxide, and hydrocarbons; and (ii) recycling at least a portion of the hydrogen stream to the methanol reactor.

18. A process for producing methanol comprising:
(a) reacting, via a catalytic partial oxidation (CPO) reaction, a CPO reactant mixture in a CPO reactor to produce a CPO reactor effluent; wherein the CPO reactant mixture comprises hydrocarbons and oxygen; wherein the CPO reactor comprises a CPO catalyst; wherein the CPO reactor effluent comprises hydrogen, carbon monoxide, carbon dioxide, water, and unreacted hydrocarbons;
(b) removing at least a portion of the carbon dioxide from the CPO reactor effluent in a carbon dioxide separator to yield syngas, wherein the syngas comprises carbon dioxide in an amount from about 0.1 mol % to about 5 mol %;
(c) introducing at least a portion of the syngas to a methanol reactor to produce a methanol reactor effluent stream; wherein the methanol reactor effluent stream comprises methanol, water, hydrogen, carbon monoxide, carbon dioxide, and hydrocarbons;
(d) separating at least a portion of the methanol reactor effluent stream into a crude methanol stream and a vapor stream, wherein the crude methanol stream comprises methanol and water, wherein the vapor stream comprises hydrogen, carbon monoxide, carbon dioxide, and hydrocarbons; and wherein the crude methanol stream comprises water in an amount of less than about 5 wt. %, based on the total weight of the crude methanol stream;
(e) separating at least a portion of the crude methanol stream in a distillation unit into a methanol stream and a water stream, wherein the distillation unit comprises one or more distillation columns;
(f) separating at least a portion of the vapor stream into a hydrogen stream and a residual gas stream, wherein the hydrogen stream comprises at least a portion of the hydrogen of the vapor stream, and wherein the residual gas stream comprises carbon monoxide, carbon dioxide, and hydrocarbons; and
(g) recycling at least a portion of the hydrogen stream to the methanol reactor.

19. The process of claim 18, wherein the CPO reactor includes a steam to carbon (S/C) molar ratio in the CPO reactant mixture of less than about 0.5:1, wherein the S/C molar ratio refers to the total moles of water (H20) in the reactant mixture divided by the total moles of carbon (C) of hydrocarbons in the reactant mixture; wherein a portion of the hydrocarbons in the CPO reactant mixture undergo decomposition to carbon and hydrogen, wherein at least a portion of the carbon reacts with carbon dioxide in the CPO reactor to produce carbon monoxide and/or wherein at least a portion of the carbon reacts with water in the CPO reactor to produce carbon monoxide and hydrogen.

20. The process of claim 18 further comprising the steps of: (1) cooling at least a portion of the CPO reactor effluent to yield a cooled CPO reactor effluent; (2) removing at least a portion of the water from the cooled CPO reactor effluent to yield a dehydrated CPO reactor effluent, wherein the dehydrated CPO reactor effluent comprises hydrogen, carbon monoxide, carbon dioxide, and unreacted hydrocarbons; and (3) feeding at least a portion of the dehydrated CPO reactor effluent to the carbon dioxide separator in step (b).
